(19) United States
(12) Patent Application Publication
(10) Pub. No.: US 2015/0057644 A1
(43) Pub. Date: Feb. 26, 2015

(54) STABILIZING LENTICULES USED FOR REFRACTIVE CORRECTION

(71) Applicant: Wavelight GMBH, Erlangen (DE)

(72) Inventor: Katrin Skerl, Bad Doberan (DE)

(73) Assignee: Wavelight GMBH, Erlangen (DE)

(21) Appl. No.: 14/378,616

(22) PCT Filed: Oct. 30, 2012
(86) PCT No.: PCT/EP2012/071431

§ 371 Date: Aug. 13, 2014

(51) Int. Cl. A61F 9/008; A61F 9/013

(52) U.S. Cl. CPC A61F 9/00827; A61F 9/00836; A61F 9/0136; A61F 2009/0087
USPC 606/5

(57) ABSTRACT

In certain embodiments, a method for refractive correction includes controlling, by one or more laser components, a focus of pulsed laser radiation having ultrashort pulses. A posterior incision is created with the pulsed laser radiation to form a posterior side of a lenticule. An anterior incision is created with the pulsed laser radiation to form an anterior side of the lenticule. Application of a stabilization solution to the lenticule is facilitated to stabilize the lenticule.

### TECHNICAL FIELD

The present disclosure relates generally to corneal surgical devices, and more particularly to stabilizing lenticules used for refractive correction.

### BACKGROUND

Refractive surgery uses lasers to reshape the cornea to correct refractive defects of the eye. According to some techniques, a flap of the eye is lifted to expose a portion of the cornea that is reshaped by ablation using an excimer laser. The flap is then replaced. According to other techniques, a femtosecond laser makes incisions in the cornea to create a lenticule. The lenticule is removed to reshape the cornea.

### BRIEF SUMMARY

In certain embodiments, a device for refractive correction comprises a laser device and a control computer. The laser device is configured to create a lenticule in an eye using pulsed laser radiation having ultrashort pulses. The laser device includes one or more laser components configured to control a focus of the pulsed laser radiation. The control computer is configured to instruct the one or more laser components to: create a posterior incision with the pulsed laser radiation to form a posterior side of a lenticule; create an anterior incision with the pulsed laser radiation to form an anterior side of the lenticule; and facilitate application of a stabilization solution to the lenticule to stabilize the lenticule.

In certain embodiments, a method for refractive correction includes controlling, by one or more laser components, a focus of pulsed laser radiation having ultrashort pulses. A posterior incision is created with the pulsed laser radiation to form a posterior side of a lenticule. An anterior incision is created with the pulsed laser radiation to form an anterior side of the lenticule. Application of a stabilization solution to the lenticule is facilitated to stabilize the lenticule.

In certain embodiments, a tangible computer-readable medium stores computer code for refractive correction that when executed by a computer is configured to control a focus of pulsed laser radiation having ultrashort pulses. The computer code is also configured to create a posterior incision with the pulsed laser radiation to form a posterior side of a lenticule; create an anterior incision with the pulsed laser radiation to form an anterior side of the lenticule; and facilitate application of a stabilization solution to the lenticule to stabilize the lenticule.

### BRIEF DESCRIPTION OF THE DRAWINGS

Exemplary embodiments of the present disclosure will now be described by way of example in greater detail with reference to the attached figures, in which:

FIG. 1 illustrates an example of a device configured to perform refractive correction according to certain embodiments;

FIG. 2 illustrates an example of a method for creating and stabilizing a lenticule according to certain embodiments;

FIGS. 3 through 6 illustrate examples of methods for stabilizing a lenticule according to certain embodiments;

FIG. 7 illustrates an anterior view of an example of a lenticule according to certain embodiments; and

FIG. 8 illustrates a cross-section of an example of a lenticule according to certain embodiments.

### DESCRIPTION OF EXAMPLE EMBODIMENTS

Referring now to the description and drawings, example embodiments of the disclosed apparatuses, systems, and methods are shown in detail. The description and drawings are not intended to be exhaustive or otherwise limit or restrict the claims to the specific embodiments shown in the drawings and disclosed in the description. Although the drawings represent possible embodiments, the drawings are not necessarily to scale and certain features may be simplified, exaggerated, removed, or partially sectioned to better illustrate the embodiments. In addition, certain drawings may be in schematic form.

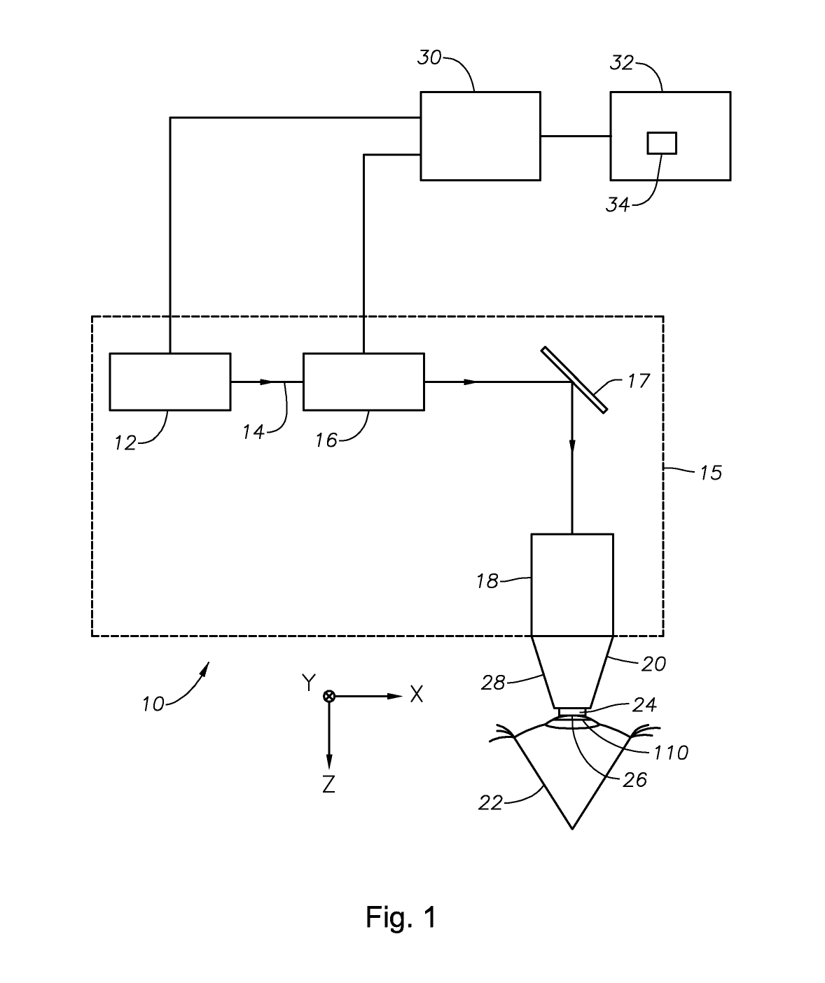

FIG. 1 illustrates an example of a device 10 configured to create and stabilize a lenticule according to certain embodiments. In the embodiments, the device 10 includes a laser device and a control computer. The laser device can create a lenticule in the cornea (such as the stroma) of an eye using pulsed laser radiation with ultrashort pulses (such as nano-, pico-, femto-, or attosecond pulses). The lenticule may be shaped according to a refractive correction profile such that when the lenticule is removed the refractive correction is applied.

The laser device may include laser components that focus the pulsed laser radiation. The control computer instructs the laser components to focus the pulsed laser radiation at the cornea to: create a posterior incision with the pulsed laser radiation to form a posterior side of the lenticule; create an anterior incision with the pulsed laser radiation to form an anterior side of a lenticule; and facilitate application of a stabilization solution to the lenticule to stabilize the lenticule. The stabilization solution is applied to stabilize the corneal tissue, which may assist with the removal of the lenticule.

The application of the stabilization solution may be facilitated in any suitable manner. For example, a channel that can be used to apply the stabilization solution may be created. The channel may be a channel that may also be used to assist in the removal of the lenticule, such as an anterior channel, a posterior channel, or a removal incision. As another example, a lenticule marking that marks a perimeter of the lenticule may be created, and the stabilization solution may be applied at the lenticule marking. As yet another example, the posterior incision may be created such that it can receive the stabilization solution. As yet another example, the anterior incision may be created such that it can receive the stabilization solution, which may facilitate separation of the lenticule from a flap.

In the illustrated example of FIG. 1, the device 10 performs surgery on the tissue of an eye 22 to create a lenticule 110. The device 10 includes a laser device 15, a patient adapter 20, a control computer 30, and a memory 32 coupled as shown according to the example. The laser device 15 may include a laser source 12, a scanner 16, one or more optical elements 17, and/or a focusing objective 18 coupled as shown according to the example. The patient adapter 20 may include a contact element 24 (which has an abutment face 26 disposed outwardly from a sample) and a sleeve 28 coupled as shown according to the example. The memory 32 stores a control program 34.

The laser source 12 generates a laser beam 14 with ultrashort pulses. In this document, an “ultrashort” pulse of light refers to a light pulse that has a duration that is less than a nanosecond, such as on the order of a nanosecond, picosecond, femtosecond, or attosecond. The focal point of the laser beam 14 may create a laser-induced optical breakdown (LIOB) in tissues such as the cornea. The laser beam 14 may be precisely focused to allow for precise incisions in the corneal cell layers, which may reduce or avoid unnecessary destruction of other tissue.

Examples of laser source 12 include nanosecond, femtosecond, picosecond, and attosecond lasers. The laser beam 14 may have any suitable wavelength, such as a wavelength in the range of 300 to 1500 nanometers (nm), for example, a wavelength in the range of 300 to 650, 650 to 1050, 1050 to 1250, or 1100 to 1500 nm. The laser beam 14 may also have a relatively small focus volume, e.g., 20 micrometers (pm) or less, such as 5 μm to 10 μm in diameter. In certain embodiments, the laser source 12 and/or delivery channel may be in a vacuum or near vacuum, e.g., less than 100 mbar.

The scanner 16, optical elements 17, and focusing objective 18 are in the beam path. The scanner 16 transversely and longitudinally controls the focal point of the laser beam 14. “Transverse” refers to a direction at right angles to the direction of propagation of the laser beam 14, and “longitudinal” refers to the direction of beam propagation. The transverse plane may be designated as the x-y plane, and the longitudinal direction may be designated as the z-direction. In certain embodiments, the abutment face 26 of the patient adapter 20 is on an x-y plane.

The scanner 16 may transversely direct the laser beam 14 in any suitable manner. For example, the scanner 16 may include a pair of galvanometrically actuated scanner mirrors that can be tilted about mutually perpendicular axes. As another example, the scanner 16 may include an electro-optical crystal that can electro-optically steer the laser beam 14. The scanner 16 may longitudinally direct the laser beam 14 in any suitable manner. For example, the scanner 16 may include a longitudinally adjustable lens, a lens of variable refractive power, or a deformable mirror that can control the z-position of the beam focus. The focus control components of the scanner 16 may be arranged in any suitable manner along the beam path, e.g., in the same or different modular units.

One (or more) optical elements 17 direct the laser beam 14 towards the focusing objective 18. An optical element 17 may be any suitable optical element that can reflect, refract, and/or diffract the laser beam 14. For example, an optical element 17 may be an immovable deviating mirror. The focusing objective 18 focuses the laser beam 14 onto the patient adapter 20, and may be separably coupled to the patient adapter 20. The focusing objective 18 may be any suitable optical element that can focus laser radiation, such as an f-theta objective.

Patient adapter 20 interfaces with the cornea of the eye 22. In the example, the patient adapter 20 has a sleeve 28 coupled to a contact element 24. The sleeve 28 couples to the focusing objective 18. The contact element 24 may be translucent or transparent to the laser radiation and has an abutment face 26 that interfaces with the cornea and may level a portion of the cornea. In certain embodiments, the abutment face 26 is planar and forms a planar area on the cornea. The abutment face 26 may be on an x-y plane, so the planar area is also on an x-y plane. In other embodiments, the abutment face 26 need not be planar, e.g., may be convex or concave.

The control computer 30 controls laser components, e.g., the laser source 12, scanner 16, and one or more optical elements 17 in accordance with the control program 34. The control program 34 contains computer code that instructs the laser components to focus the pulsed laser radiation at a region of the cornea to photodisrupt at least a portion of the region.

In certain examples of operation, the scanner 16 may direct the laser beam 14 to form incisions of any suitable geometry. Examples of types of incisions include planar incisions and lateral incisions. A planar incision is two-dimensional incision that is typically on an x-y plane. The scanner 16 may form a planar incision by focusing the laser beam 14 at a constant z-value under the abutment face 26 and moving the focus in a pattern in an x-y plane. A curved planar incision may be formed in a similar matter, but varying the z-value to curve the planar area. A lateral incision is an incision that extends from under the corneal surface (such as from a planar incision) to the surface. The scanner 16 may form a lateral incision by changing the z-value of the focus of the laser beam 14 and optionally changing the x and/or y values.

Any suitable portion of the cornea may be photodisrupted, and one or more of any of the corneal layers may be selected for photodisruption. The device 10 may photodisrupt a corneal layer in any suitable manner. In certain embodiments, the control computer 30 may instruct the laser device to focus the laser beam 14 at a constant z-value under the abutment face 26 and move in a pattern in the x-y plane that substantially covers a target zone selected for photodisruption. Any suitable pattern may be used. For example, according to a meander pattern or line pattern, the scan path has a constant y-value and moves in the +x direction. When the scan path reaches a point of the border of the target zone, the path moves to a next y value that is a predetermined distance from the previous y-value and then moves in the −x direction until it reaches another point of the border. The scan path continues until the entire target zone is scanned. As another example, according to a spiral pattern or curved pattern, the scan path starts at or near the center of the target zone and moves in a spiral or concentric circle pattern until the path reaches the border of the target zone, or vice-versa.

As the laser beam 14 travels along the scan path, the laser beam pulses create disruptions, such as microdisruptions. In certain situations, a scan path pattern may yield a non-uniform distribution of microdisruptions over the target zone. In these cases, the laser beam 14 may be modified to make the distribution more uniform. For example, certain pulses may be blocked or the pulse energy may be decreased to reduce number of or the effect of the pulses in a particular region.

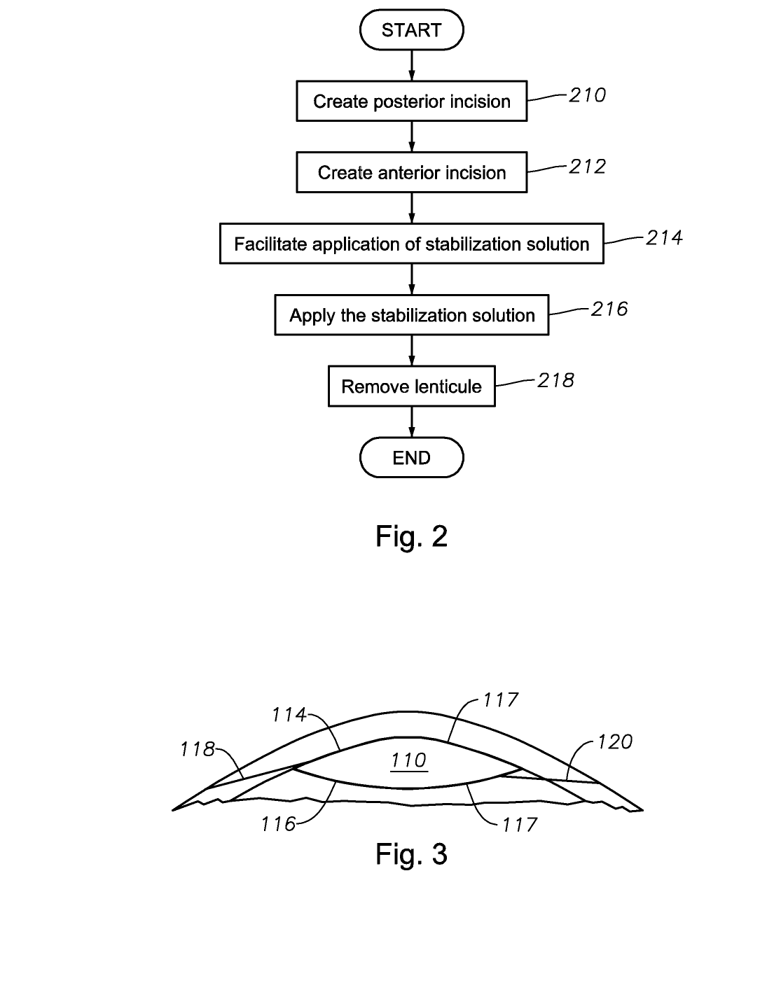

FIG. 2 illustrates an example of a method for creating and stabilizing a lenticule in a cornea of an eye according to certain embodiments. The method may be performed by the system 10 of FIG. 1. The method starts at step 210, where a posterior incision 116 is created. The posterior incision 116 forms a posterior surface of the lenticule 110. An anterior incision 114 is created at step 212. The anterior incision 114 forms an anterior surface of the lenticule 110. Application of a stabilization solution is facilitated at step 214. The application of a stabilization solution may be facilitated in any suitable manner. Examples of facilitating application of stabilization solution are described in more detail with reference to FIGS. 3 through 6.

The stabilization solution is applied at step 216 to strengthen corneal tissue, e.g., the lenticule 110. The stabilization solution may stabilize corneal tissue in any suitable manner, e.g., by encouraging cross-linking. The stabilization solution may comprise any suitable ingredients that stabilize corneal tissue, e.g., riboflavin (vitamin B2), lysyloxidase, transglutaminase, sugar aldehydes, ethylcarbodiimid, glutaraldehyde, formaldehyde, or combinations of two or more of any of the preceding, e.g., a Karnovsky solution. The stabilization solution may be manually or automatically applied. The lenticule 110 is removed at step 218. The lenticule 110 may be manually or automatically removed through a removal incision or other channel.

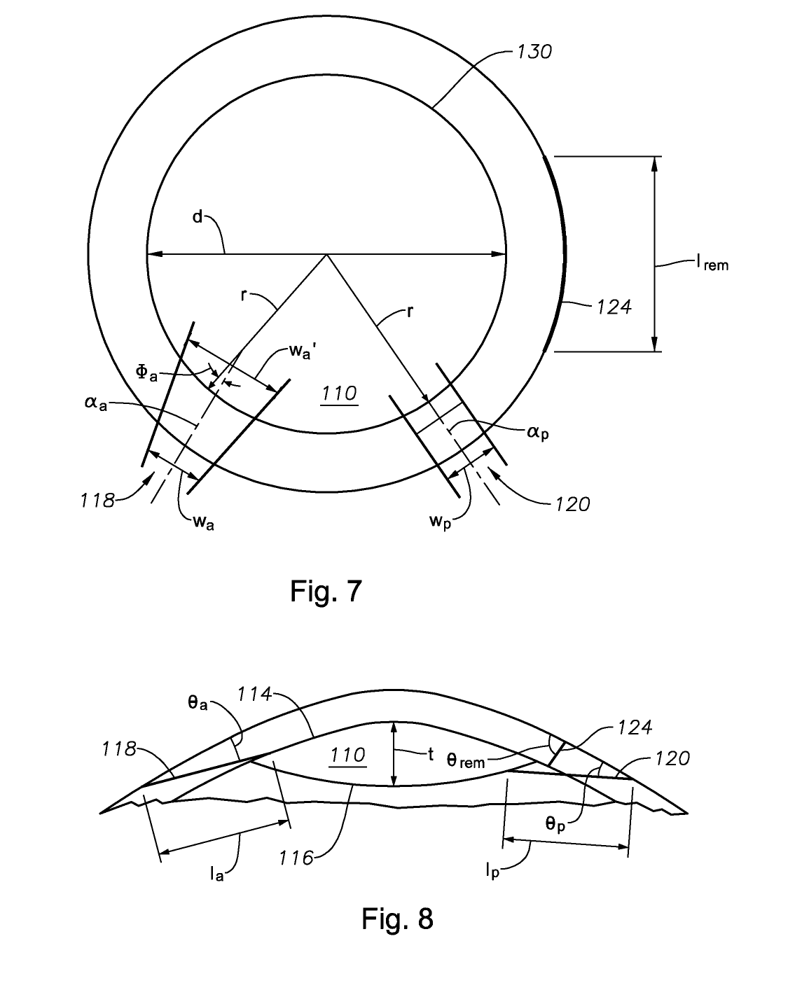

FIGS. 3 through 6 illustrate examples of facilitating application of the stabilization solution. FIG. 3 illustrates creating an anterior incision 114 and posterior incision 116 to form a lenticule 110, and then applying the stabilization solution 117 to the anterior and/or posterior sides of the lenticule 110. The solution may be applied through one or more channels, e.g., channel 118, channel 120, a removal incision, and/or tissue separated by photodisruption. The solution may travel through a channel, incision, or tissue separation via the capillary effect. Examples of channels are described in more detail with reference to FIGS. 7 and 8.

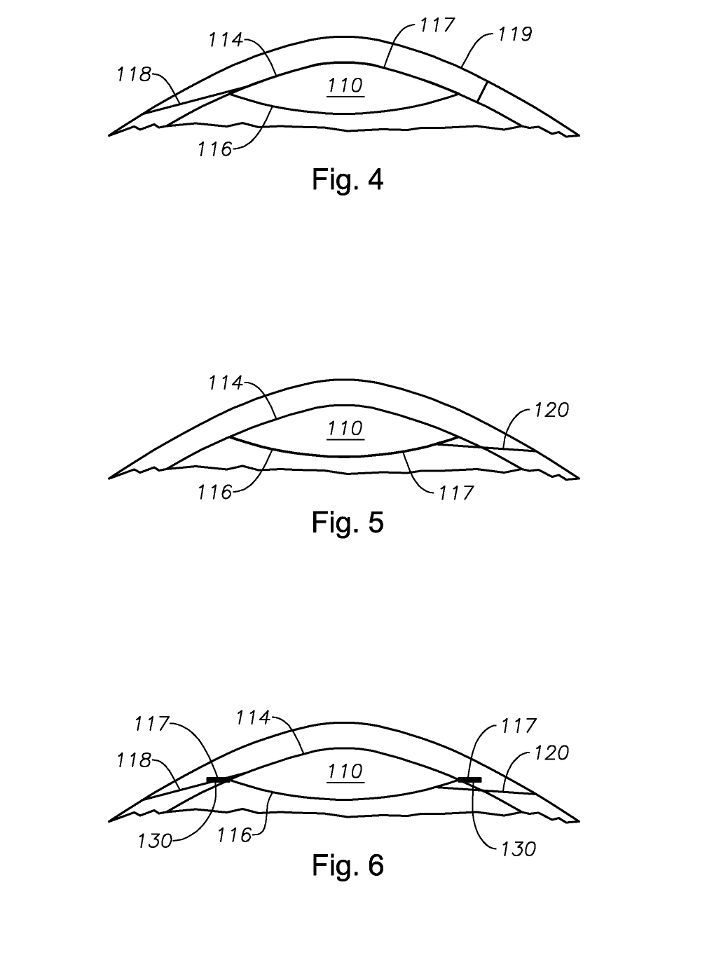

FIG. 4 illustrates creating an anterior incision 114, a posterior incision 116, and/or a flap 119 and then applying the stabilization solution 117 to the anterior side of the lenticule 110, e.g., prior to lifting the flap 119. The solution may be applied through the channel 118.

FIG. 5 illustrates creating an anterior incision 114 and posterior incision 116 to form a lenticule 110 (which may be very thin, e.g., 3 μm to 7 μm thick, such as approximately 5 μm thick), and then applying the stabilization solution 117 to the posterior side of the lenticule 110. The solution may be applied through a channel 120.

FIG. 6 illustrates creating an anterior incision 114, a posterior incision 116, and a lenticule marking 130 that marks a perimeter of the lenticule 110. The stabilization solution 117 is applied at the lenticule marking 130. Examples of lenticule markings are described in more detail with reference to FIG. 7. The solution may be applied through a channel 118 and/or 120.

FIGS. 7 and 8 illustrate an example of creating a lenticule 110, as well as an anterior channel 118, a posterior channel 120, and/or a removal incision 124, according to certain embodiments. FIG. 7 illustrates an anterior view of the lenticule 110, and FIG. 8 illustrates a cross-section of the lenticule 110.

The lenticule 110 may have any suitable shape. In certain embodiments, the lenticule 110 may have a flattened, disc shape with any suitable perimeter, e.g., a circular, elliptical, free form, or irregular. The lenticule 110 may have any suitable size. For example, the lenticule 110 may have any suitable diameter d (or radius r), such as a diameter d in the range of 1 to 10 mm, 3 to 8 mm, or 5 to 7 mm, such as approximately 6.5 mm. The lenticule 110 may have any suitable thickness t, such as a value in the range of 3 to 200 micrometers (μm), 10 to 100 μm, and 40 to 60 μm, such as approximately 50 μm.

The device 10 may create the lenticule 110 in any suitable manner. In certain embodiments, the control computer 30 may instruct the laser device to create a posterior incision 116 and an anterior incision 114, which are types of curved planar incisions, using laser radiation. The anterior incision 114 forms the anterior side of the lenticule 110, and the posterior incision 116 forms the posterior side of the lenticule 110. In certain embodiments, the anterior incision 114 and/or posterior incision 116 yields a refractive profile for refractive correction such that a refractive correction is applied after removal of the lenticule 110.

The anterior 114 and posterior 116 incisions may be created in any suitable order and in any suitable manner. In certain embodiments, a channel, which may be a type of lateral incision, may facilitate removal of the lenticule 110. For example, an anterior channel 118 may be used to separate the anterior side of the lenticule 110 from the surrounding tissue, and/or a posterior channel 120 may be used to separate the posterior side of the lenticule 110 from the surrounding tissue. In the embodiments, the channel may be used to insert (e.g., manually or automatically) an instrument into an incision to separate a surface of the lenticule 110 from the rest of the cornea in order to remove the lenticule 110.

The channels and incisions may be created in any suitable order. For example, a channel may be created before or after the corresponding incision. As another example, an anterior channel and/or anterior incision may be created before or after a posterior channel and/or posterior incision. In certain embodiments, the posterior channel 120 is created, and then a posterior incision 116 is created. An anterior channel 118 is created, and then an anterior incision 114 is created.

A channel may have any suitable size and shape. In certain embodiments, a channel with a center line αᵢ, where i identifies the channel, may have any suitable length lᵢ, width wᵢ, angle φᵢ of center line αᵢ with respect to radius r, and angle θᵢ of center line αᵢ with respect to the anterior surface of the eye. In FIG. 7, the anterior channel 118 has a narrower width wₐ towards the entrance of the channel and a wider width wₐ′ towards the center of lenticule 110. The posterior channel 120 has the same width wₚ from end to end. The widths may have any suitable value, such as a value in the range 0.5 to 4, 1 to 3, or 1.5 to 2.0 mm. In other examples, the posterior channel may be shaped like channel 118 or may have any other suitable shape, and the anterior channel may be shaped like channel 120 or may have any other suitable shape. The posterior and anterior channels may have the same shape or may have different shapes. The center line αₐ of the anterior channel 118 is at an angle φₐ with respect to radius r. The center line αₚ of the posterior channel 120 is at an angle φₚ (not labeled) of 0° with respect to radius r. The angles φᵢ may have any suitable value, such as a value in the range 0 to 5, 5 to 10, 10 to 15, or 15 to 20 degrees.

Referring to FIG. 8, the anterior channel 118 has length lₐ, and the posterior channel 120 has length lₚ. The lengths may have any suitable value, such as a value in the range 1 to 5 mm. The center line αₐ of anterior channel 118 has an angle θₐ with respect to the surface of the eye, and the center line αₚ of posterior channel 120 has an angle θₚ. The angles θᵢ may have any suitable value, such as a value where the channel is substantially tangential or nearly tangential (within 5 degrees) to the corresponding incision to allow for an instrument that is inserted into the channel to enter the incision and separate a surface of the lenticule from the rest of the cornea. For example, angles θᵢ may have a value in the range 0 to 10, 10 to 20, or 20 to 30 degrees, which may allow the channels to be tangential or nearly tangential to a surface of the lenticule. In certain embodiments, the angles θᵢ may have different values at the entrance of the eye (e.g., approximately 90 degrees) and then change to values that allow the channels to be tangential or nearly tangential to a surface of the lenticule.

Referring to FIG. 7, a lenticule marking 130 can be created by the laser device. The lenticule marking 130 may have any suitable dimensions, where the dimensions may describe the shape (e.g., perimeter shape), size (e.g., diameter), and location (e.g., x, y, z position) of the lenticule marking 130. In certain embodiments, the lenticule marking 130 may have a perimeter shape that substantially matches, e.g., is substantially the same shape as, the lenticule perimeter shape. In these embodiments, the lenticule marking 130 may serve as an outline of the lenticule 110. In certain embodiments, the laser device may form a removal incision 124 through which the lenticule 110 may be manually or automatically extracted. The removal incision 124 may have any suitable size or shape. In certain embodiments, the removal incision 124 may have any suitable length lᵣₑₘ and angle θᵣₑₘ with respect to the surface of the eye. For example, length lᵣₑₘ may have a value that allows for the lenticule 110 to be extracted through it, such as a value that is approximately the size of diameter d, but perhaps up to 2 mm larger or smaller. Angle θᵣₑₘ may have a value in the range of 80 to 110 degrees.

A component (such as the control computer 30) of the systems and apparatuses disclosed herein may include an interface, logic, memory, and/or other suitable element, any of which may include hardware and/or software. An interface can receive input, send output, process the input and/or output, and/or perform other suitable operations. Logic can perform the operations of a component, for example, execute instructions to generate output from input. Logic may be encoded in memory and may perform operations when executed by a computer. Logic may be a processor, such as one or more computers, one or more microprocessors, one or more applications, and/or other logic. A memory can store information and may comprise one or more tangible, computer-readable, and/or computer-executable storage medium. Examples of memory include computer memory (for example, Random Access Memory (RAM) or Read Only Memory (ROM)), mass storage media (for example, a hard disk), removable storage media (for example, a Compact Disk (CD) or a Digital Video or Versatile Disk (DVD)), database and/or network storage (for example, a server), and/or other computer-readable media.

In particular embodiments, operations of the embodiments may be performed by one or more computer readable media encoded with a computer program, software, computer executable instructions, and/or instructions capable of being executed by a computer. In particular embodiments, the operations may be performed by one or more computer readable media storing, embodied with, and/or encoded with a computer program and/or having a stored and/or an encoded computer program.

Although this disclosure has been described in terms of certain embodiments, modifications (such as changes, substitutions, additions, omissions, and/or other modifications) of the embodiments will be apparent to those skilled in the art. Accordingly, modifications may be made to the embodiments without departing from the scope of the invention. For example, modifications may be made to the systems and apparatuses disclosed herein. The components of the systems and apparatuses may be integrated or separated, and the operations of the systems and apparatuses may be performed by more, fewer, or other components. As another example, modifications may be made to the methods disclosed herein. The methods may include more, fewer, or other steps, and the steps may be performed in any suitable order.

Other modifications are possible without departing from the scope of the invention. For example, the description illustrates embodiments in particular practical applications, yet other applications will be apparent to those skilled in the art. In addition, future developments will occur in the arts discussed herein, and the disclosed systems, apparatuses, and methods will be utilized with such future developments.

The scope of the invention should not be determined with reference to the description. In accordance with patent statutes, the description explains and illustrates the principles and modes of operation of the invention using exemplary embodiments. The description enables others skilled in the art to utilize the systems, apparatuses, and methods in various embodiments and with various modifications, but should not be used to determine the scope of the invention.

The scope of the invention should be determined with reference to the claims and the full scope of equivalents to which the claims are entitled. All claims terms should be given their broadest reasonable constructions and their ordinary meanings as understood by those skilled in the art, unless an explicit indication to the contrary is made herein. For example, use of the singular articles such as “a,” “the,” etc. should be read to recite one or more of the indicated elements, unless a claim recites an explicit limitation to the contrary. As another example, “each” refers to each member of a set or each member of a subset of a set, where a set may include zero, one, or more than one element. In sum, the invention is capable of modification, and the scope of the invention should be determined, not with reference to the description, but with reference to the claims and their full scope of equivalents.

What is claimed is:

1. A method for refractive correction comprising:
controlling, by one or more laser components of a laser device, a focus of pulsed laser radiation having a plurality of ultrashort pulses;
creating a posterior incision with the pulsed laser radiation to form a posterior side of a lenticule;
creating an anterior incision with the pulsed laser radiation to form an anterior side of the lenticule; and
facilitating application of a stabilization solution to the lenticule to stabilize the lenticule.

2. The method of claim 1, further comprising:
applying the stabilization solution to the lenticule to stabilize the lenticule.

3. The method of claim 1, the facilitating application of the stabilization solution further comprising:
creating a channel that can receive the stabilization solution.

4. The method of claim 1, the facilitating application of the stabilization solution further comprising:
creating a channel that can receive the stabilization solution; and
applying the stabilization solution through the channel.

5. The method of claim 1, the facilitating application of the stabilization solution further comprising:
creating a lenticule marking that marks the application of the stabilization solution at a lenticule perimeter of the lenticule.

6. The method of claim 1, the facilitating application of the stabilization solution further comprising:
creating a lenticule marking that marks the application of the stabilization solution at a lenticule perimeter of the lenticule; and
applying the stabilization solution at the lenticule marking.

7. The method of claim 1, the facilitating application of the stabilization solution further comprising:
creating the posterior incision to receive the stabilization solution.

8. The method of claim 1, the facilitating application of the stabilization solution further comprising:
creating the posterior incision to receive the stabilization solution; and
applying the stabilization solution at the posterior incision.

9. The method of claim 1, the facilitating application of the stabilization solution further comprising:
creating the anterior incision to receive the stabilization solution.

10. The method of claim 1, the facilitating application of the stabilization solution further comprising:
creating the anterior incision to receive the stabilization solution; and
applying the stabilization solution at the anterior incision.

11. The method of claim 1, further comprising:
creating a flap to facilitate removal of the lenticule; and
facilitating application of the stabilization solution by applying the stabilization solution at the posterior incision.

12. A device for refractive correction, the device comprising:
a laser device configured to create a lenticule in an eye using pulsed laser radiation having a plurality of ultrashort pulses, the laser device comprising one or more laser components configured to control a focus of the pulsed laser radiation; and
a control computer configured to instruct the one or more laser components to:
create a posterior incision with the pulsed laser radiation to form a posterior side of the lenticule;
create an anterior incision with the pulsed laser radiation to form an anterior side of the lenticule; and
facilitate application of a stabilization solution to the lenticule to stabilize the lenticule.

13. The device of claim 12, the control computer configured to:
apply the stabilization solution to the lenticule to stabilize the lenticule.

14. The device of claim 12, the control computer configured to instruct the one or more laser components to facilitate application of the stabilization solution by:
creating a channel that can receive the stabilization solution.

15. The device of claim 12, the control computer configured to instruct the one or more laser components to facilitate application of the stabilization solution by:
creating a channel that can receive the stabilization solution; and
applying the stabilization solution through the channel.

16. The device of claim 12, the control computer configured to instruct the one or more laser components to facilitate application of the stabilization solution by:
creating a lenticule marking that marks the application of the stabilization solution at a lenticule perimeter of the lenticule.

17. The device of claim 12, the control computer configured to instruct the one or more laser components to facilitate application of the stabilization solution by:
creating a lenticule marking that marks the application of the stabilization solution at a lenticule perimeter of the lenticule; and
applying the stabilization solution at the lenticule marking.

18. The device of claim 12, the control computer configured to instruct the one or more laser components to facilitate application of the stabilization solution by:
creating the posterior incision to receive the stabilization solution.

19. The device of claim 12, the control computer configured to instruct the one or more laser components to facilitate application of the stabilization solution by:
creating the posterior incision to receive the stabilization solution; and
applying the stabilization solution at the posterior incision.

20. The device of claim 12, the control computer configured to instruct the one or more laser components to facilitate application of the stabilization solution by:
creating the anterior incision to receive the stabilization solution.

21. The device of claim 12, the control computer configured to instruct the one or more laser components to facilitate application of the stabilization solution by:
creating the anterior incision to receive the stabilization solution; and
applying the stabilization solution at the anterior incision.

22. The device of claim 12, the control computer configured to instruct the one or more laser components to:
create a flap to facilitate removal of the lenticule; and
facilitate application of the stabilization solution by applying the stabilization solution at the posterior incision.

23. One or more tangible computer-readable media storing computer code that when executed by a computer is configured to:
control, by one or more laser components of a laser device, a focus of pulsed laser radiation having a plurality of ultrashort pulses;
create a posterior incision with the pulsed laser radiation to form a posterior side of a lenticule;
create an anterior incision with the pulsed laser radiation to form an anterior side of the lenticule; and
facilitate application of a stabilization solution to the lenticule to stabilize the lenticule.

24. The computer-readable media of claim 23, further configured to:
apply the stabilization solution to the lenticule to stabilize the lenticule.

25. The computer-readable media of claim 23, further configured to facilitate application of the stabilization solution by:
creating a channel that can receive the stabilization solution; and
applying the stabilization solution through the channel.

26. The computer-readable media of claim 23, further configured to facilitate application of the stabilization solution by:
creating a lenticule marking that marks the application of the stabilization solution at a lenticule perimeter of the lenticule; and
applying the stabilization solution at the lenticule marking.

27. The computer-readable media of claim 23, further configured to facilitate application of the stabilization solution by:
creating the posterior incision to receive the stabilization solution; and
applying the stabilization solution at the posterior incision.

28. The computer-readable media of claim 23, further configured to facilitate application of the stabilization solution by:
creating the anterior incision to receive the stabilization solution; and
applying the stabilization solution at the anterior incision.

29. The computer-readable media of claim 23, further configured to:
create a flap to facilitate removal of the lenticule; and
facilitate application of the stabilization solution by applying the stabilization solution at the posterior incision.
